(19) United States
(12) Patent Application Publication
(10) Pub. No.: US 2013/0284179 A1
(43) Pub. Date: Oct. 31, 2013

(54) REMOVAL OF CARBON DIOXIDE FROM PATIENT EXPIRED GAS DURING ANESTHESIA

(71) Applicant: Tofy MUSSIVAND, Ottawa (CA)

(72) Inventor: Tofy MUSSIVAND, Ottawa (CA)

(21) Appl. No.: 13/458,325

(22) Filed: Apr. 27, 2012

(51) Int. Cl. A61M 16/22

(52) U.S. Cl. 128/205.28

(57) ABSTRACT

An assembly (10) for removing carbon dioxide from patient expired gas during anesthesia is described. The assembly (10) comprises a chamber (12) configured to contain an adsorbent (14) for treating the patient expired gas during a capture mode of operation and return treated patient expired gas to an anesthesia machine; integral regeneration equipment (28, 30, 44) configured to regenerate the adsorbent (14) during a release mode of operation; and a control device for switching from the capture mode of operation to the release mode of operation.

### TECHNICAL FIELD

The disclosure relates generally to anesthesia systems, and more particularly to devices, systems and methods for the removal of carbon dioxide from patient expired gas during anesthesia.

### BACKGROUND OF THE ART

During surgery where a patient is under general anesthesia, an anesthesia breathing machine may be used to administer an anesthetic agent to the patient and may also serve as a respiratory device (e.g. ventilator) by supplying oxygen to the patient. It is desirable that such anesthesia machines operate in closed or semi-closed circuits in order to recycle the anesthetic agent that is expired by the patient by causing a portion of the patient expired gas to be re-breathed. In these circuits, it is critical that carbon dioxide (CO₂) be removed from the patient expired gas prior to re-breathing to prevent a buildup that could cause metabolic acidosis and/or hypoxia in the patient and also that the patient be provided with an adequate supply of oxygen.

Existing methods for removing CO₂ from patient expired gas during anesthesia include the use of an absorbent in pellet form in a canister in the breathing circuit. However, as the absorbent is consumed, its efficiency becomes degraded and can lead to increased levels of CO₂ in the breathing circuit. When an absorbent is completely consumed, it must be discarded as medical waste. Absorbents can also cause a temperature rise within the canister during absorption of CO₂. Additionally, some absorbents can react with anesthetic agents and produce toxic byproducts within the breathing circuit including carbon monoxide, compound A, methanol and formaldehyde.

Another method for CO₂ removal includes the use of an adsorbent which may be regenerated after use. This method may require the adsorbent to be sent off-site to be regenerated since the regeneration equipment necessary is typically not found at or near areas where anesthesia machines are used (e.g. operating room).

Improvement in the removal of CO₂ from patient expired gas during anesthesia is therefore desirable.

### SUMMARY

The disclosure describes systems, devices, and methods for removing CO₂ from patient expired gas during anesthesia. The systems, devices and methods may permit use of an adsorbent for the removal of CO₂ from patient expired gas during anesthesia and also permit regeneration of the adsorbent without requiring the adsorbent to be sent off-site.

Thus, in one aspect, the disclosure describes a standalone assembly for removing carbon dioxide from patient expired gas during anesthesia. The assembly may comprise: a chamber configured to contain an adsorbent for treating the patient expired gas, the chamber including a first inlet for receiving the patient expired gas for treatment by the adsorbent and a first outlet for returning treated patient expired gas to an anesthesia machine during a capture mode of operation; regeneration equipment configured to regenerate the adsorbent during a release mode of operation; and a control device for switching from the capture mode of operation to the release mode of operation.

In another aspect, the disclosure describes a method for removing carbon dioxide from patient expired gas during anesthesia using a standalone assembly wherein the assembly comprises a chamber containing an adsorbent and regeneration equipment for regenerating the adsorbent. The method may comprise: during a capture mode of operation of the assembly: receiving the patient expired gas into the chamber; removing carbon dioxide from the patient expired gas using the adsorbent; and returning treated patient expired gas to an anesthesia machine; switching from a capture mode of operation to a release mode of operation; and activating the regeneration equipment integral to the assembly to regenerate the adsorbent.

In another aspect, the disclosure describes a system for removing carbon dioxide from patient expired gas during anesthesia wherein the system is connectable in line with an expiratory limb of an anesthesia machine. The system may comprise: a canister for containing an adsorbent for treating the patient expired gas, the canister including a first inlet for receiving the patient expired gas and a first outlet for returning treated patient expired gas to the anesthesia machine during a capture mode of operation, the canister including a second inlet for receiving a regenerative fluid for regenerating the adsorbent and a second outlet for releasing the regenerative fluid, after the regenerative fluid has at least partially regenerated the adsorbent, during a release mode of operation; a fluid propeller integral to the canister and configured to induce a flow of regenerative fluid through the second inlet and out of the second outlet for regenerating the adsorbent during a release mode of operation; and a control device configured to switch from the capture mode of operation to the release mode of operation.

In another aspect, the disclosure describes an assembly for removing carbon dioxide from patient expired gas during anesthesia wherein the assembly is connectable in line with an expiratory limb of an anesthesia machine. The system may comprise: a chamber for containing an adsorbent for treating the patient expired gas, the chamber including a first inlet for receiving the patient expired gas for treatment by the adsorbent and a first outlet for returning treated patient expired gas to the anesthesia machine during a capture mode of operation, the chamber including a second inlet for receiving a flow of air from an ambient environment for regenerating the adsorbent and a second outlet for releasing the air to the ambient environment, after the air has come in contact with the adsorbent, to regenerate the adsorbent during a release mode of operation; a filter configured to filter the air being released to the ambient environment through the second outlet during the release mode of operation, the filter and the chamber being integrated in a common support structure; and a control device configured to switch from the capture mode of operation to the release mode of operation.

In another aspect, the disclosure describes a system for removing carbon dioxide from patient expired gas during anesthesia wherein the system is connectable in line with an expiratory limb of an anesthesia machine. The system may comprise: a chamber for containing an adsorbent for treating the patient expired gas, the chamber including a first inlet for receiving the patient expired gas for treatment by the adsorbent and a first outlet for returning treated patient expired gas to the anesthesia machine during a capture mode of operation, the chamber including a second inlet for receiving a regenerative fluid for at least partially regenerating the adsorbent and a second outlet for releasing the regenerative fluid, after the regenerative fluid has at least partially regenerated the adsorbent, during a release mode of operation; a fluid propeller configured to induce a flow of regenerative fluid through the second inlet and the second outlet for regenerating the adsorbent during a release mode of operation; and a control device configured to switch from the capture mode of operation to the release mode of operation, the control device including at least one of a first flow control device to simultaneously occlude the first inlet and the first outlet and a second flow control device to simultaneously open the second inlet and the second outlet.

In another aspect, the disclosure describes a system for removing carbon dioxide from patient expired gas during anesthesia. The assembly may comprise: a chamber configured to contain an adsorbent for treating the patient expired gas, the chamber including a first inlet for receiving the patient expired gas for treatment by the adsorbent and a first outlet for returning treated patient expired gas to an anesthesia machine during a capture mode of operation; regeneration equipment configured to regenerate the adsorbent during a release mode of operation, the regeneration device and the chamber being integrated in a common support structure; and a control device for switching from the capture mode of operation to the release mode of operation.

In a further aspect, the disclosure describes a system for removing carbon dioxide from patient expired gas during anesthesia wherein the system is connectable in line with an expiratory limb of an anesthesia machine. The system may comprise: a chamber for containing an adsorbent and configured to receive the patient expired gas from the expiratory limb of the anesthesia machine and produce treated patient expired gas for returning to the anesthesia machine during a capture mode of operation, the chamber being configured to receive a regenerative fluid during a release mode of operation; regeneration equipment configured to regenerate the adsorbent during the release mode of operation; and a control device for switching from the capture mode of operation to the release mode of operation, the control device being configured to substantially prevent the patient expired gas from entering the chamber and permit the regenerative fluid to flow through the chamber, the control device comprising at least one flow control device actuatable from at least one of an open and a closed position in response to an applied force greater than a magnetic attraction holding the at least one flow control device in the at least one open position and closed position; wherein in the open position, the at least one flow control device permits a flow of one of patient expired gas and regenerative fluid through the chamber, and in the closed position, the at least one flow control device substantially prevents the flow of one of patient expired gas and regenerative fluid through the chamber.

Further details of these and other aspects of the subject matter of this application will be apparent from the detailed description and drawings included below.

### DESCRIPTION OF THE DRAWINGS

Reference is now made to the accompanying drawings, in which:

FIG. 1 is an isometric view schematically showing an assembly for use in removing CO₂ from patient expired gas(es) during anesthesia, with adsorbent material removed for clarity;

FIG. 2 is a cross-sectional view of the assembly of FIG. 1 along line 2-2 in FIG. 1;

FIG. 3 is an isometric exploded view of components within the assembly of FIG. 1;

FIGS. 4A and 4B are cross-sectional views taken along line 4-4 in FIG. 1 of a flow control device in an open and closed position respectively;

FIGS. 5A and 5B are cross-sectional views taken along line 5-5 in FIG. 1 of the flow control device of FIGS. 4A and 4B in an open and closed position respectively;

FIG. 6 is a cross-sectional view of the assembly of FIG. 1 along line 6-6 in FIG. 1 where a filter element is partially withdrawn;

FIG. 7 is top view of the filter element of FIG. 6 partially extending across an outlet passage of the assembly of FIG. 1;

FIG. 8 is an isometric view of a gas distributor of the assembly of FIG. 1;

FIG. 9 is an isometric exploded view of an arrangement for coupling the assembly of FIG. 1 to an expiratory line of an anesthesia machine;

FIG. 10 is an isometric view schematically showing the assembly of FIG. 1 connected to an expiratory line of an anesthesia machine circuit and showing a flow of patient expired gas(es) during a capture mode of operation, with the adsorbent material removed for clarity;

FIG. 11 is a cross-sectional view of the assembly of FIG. 10 taken along line 11-11 in FIG. 10 showing the flow of patient expired gas(es) during a capture mode of operation;

FIG. 12 is an isometric view schematically showing the assembly of FIG. 1 connected to an expiratory line of an anesthesia machine circuit and showing a flow of regenerative fluid(s) during a release mode of operation, with the adsorbent material removed for clarity;

FIG. 13 is a cross-sectional view of the assembly of FIG. 12 taken along line 13-13 in FIG. 12 showing the flow of regenerative fluid(s) during a release mode of operation; and

FIG. 14 is a schematic representation of an arrangement for semi-automatically and/or automatically switching the assembly of FIG. 1 between the capture mode of operation and the release mode of operation.

### DETAILED DESCRIPTION OF THE PREFERRED EMBODIMENTS

Various aspects of preferred embodiments are described through reference to the drawings.

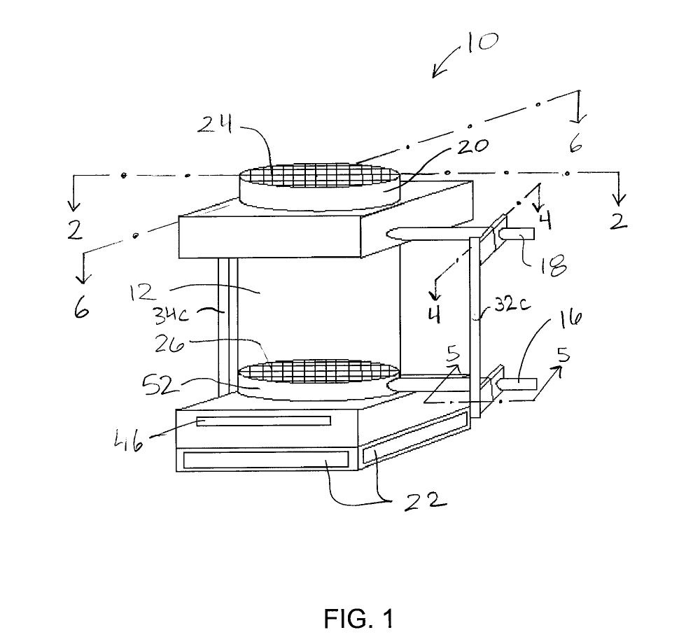

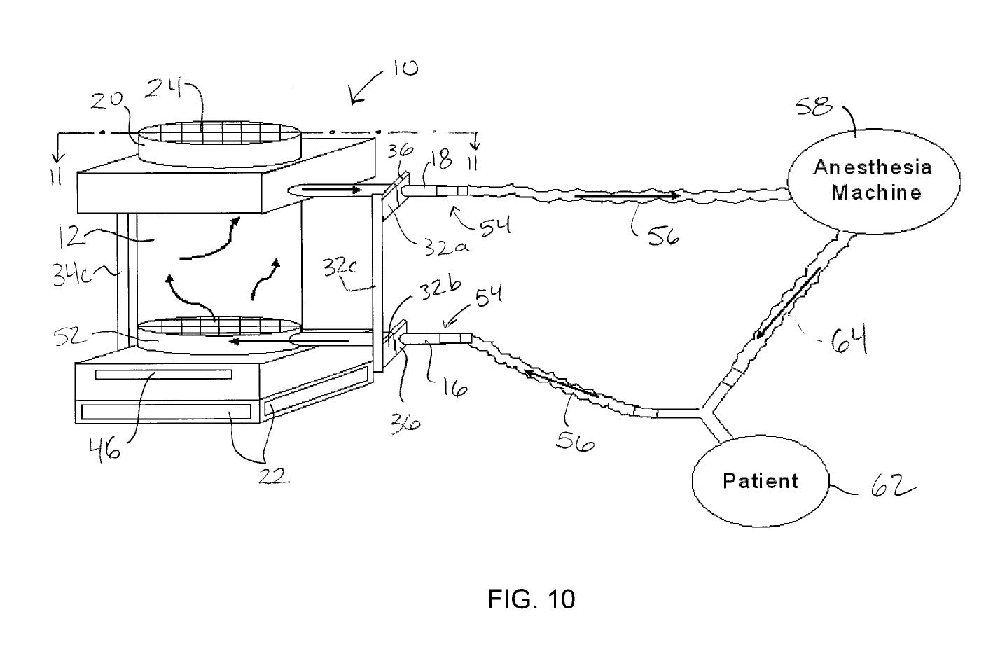

FIG. 1 schematically illustrates an assembly, generally shown at 10, for removing CO₂ from patient expired gas(es) during anesthesia. Assembly 10 may be a standalone (e.g. self-contained) and reusable CO₂ removal system which makes use of at least one adsorbent. The adsorbent(s) is/are not shown in FIG. 1. Assembly 10 may comprise integral regeneration equipment configured to regenerate the adsorbent(s) when required. Assembly 10 may be configured for in-line connection to an expiratory limb of an anesthesia machine as illustrated in FIGS. 10 and 12 and explained further below. Accordingly, assembly 10 may be used for removing CO₂ from patient expired gas during anesthesia during a capture mode of operation and the regeneration equipment integral to assembly 10 may be used to regenerate the adsorbent during a release mode of operation such as, for example after anesthesia and/or surgery. Assembly 10 may be substantially self-contained so that it may be able to operate in the capture mode and/or release mode without any auxiliary equipment being required. While the following disclosure relates mainly to anesthesia, it is understood that assembly 10 could also be used in other applications and in conjunction with other devices such as ventilators, respirators and breathing apparatuses for medical procedures, under water diving and/or aerospace breathing for example.

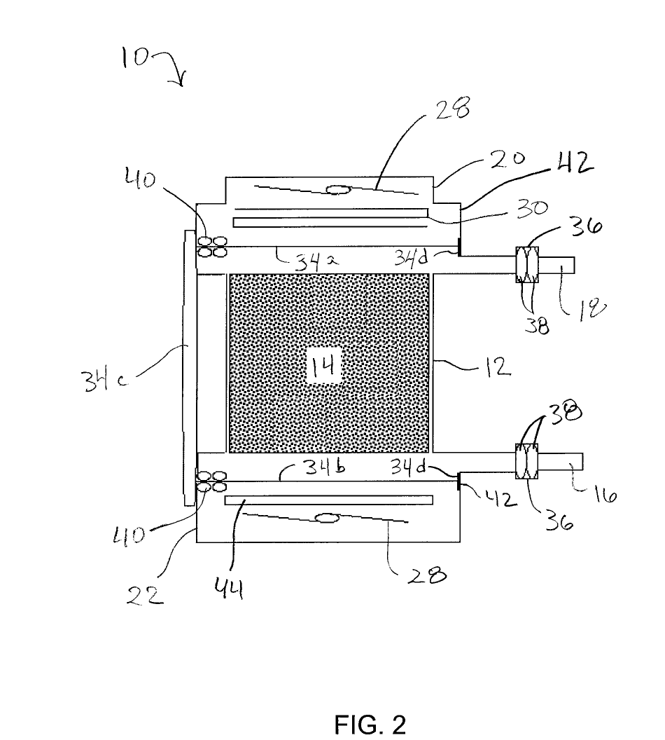

Assembly 10 may comprise chamber(s) 12, within a canister for example, configured to contain adsorbent material(s) 14 (shown in FIG. 2) for treating (e.g. capturing) patient expired gas(es). Chamber(s) 12 (e.g. canister) and at least some regeneration equipment associated with assembly 10 may be integrated in a common support structure so that assembly 10 may be substantially self-contained. Accordingly, regeneration equipment may be directly or indirectly attached to a canister containing chamber(s) 12 so that assembly 10 may be transported as a unit. For example, assembly 10 may be relatively portable so that it may be transported from one operating room to another. Alternatively, transportation of assembly 10 may not be required since regeneration of adsorbent(s) 14 may take place on site without physically moving assembly 10 away from a breathing circuit.

Adsorbent(s) 14 may comprise any suitable CO₂ adsorbent(s). For example, adsorbent(s)14 may include any one of the following materials: Mobil's Composition of Matter No. 41 (MCM-41); pore-expanded MCM-41 (PE-MCM-41), triamine surface modified MCM-41 (TRI-PE-MCM-41); Mobil's Composition of Matter No. 48 (MCM-48); Santa Barbara Amorphous Type 1 (SBA-1); Santa Barbara Amorphous Type 15 (SBA-15); activated carbon (i.e. anthracite), lithium silicate (Li₄SiO₄) and various Zeolites such as 13×. Adsorbent(s) 14 may be in the form of a bed of particles (e.g. pellets) through which patient expired gas(es) may flow. An adsorbent that does not contain relatively strong bases may be selected to reduce the risk of generating toxic compounds during the capture and/or release mode of operation.

Chamber(s) 12 may include first inlet(s) 16 for receiving the patient expired gas(es) and first outlet(s) 18 for returning treated patient expired gas(es) to an anesthesia machine during a capture mode of operation. Chamber(s) 12 may include second inlet(s) 20 for receiving regenerative fluid(s) and second outlet(s) 22 for releasing the regenerative fluid(s) during a release mode of operation where the regenerative fluid(s) may come in contact with adsorbent(s) 14 by flowing through chamber(s) 12. Inlets 16, 20 and outlets 18, 22 may each comprise one or more inlet/outlet passages to/from chamber(s) 12. Regenerative fluid(s) may include, for example, a source nitrogen gas, which may be heated and/or ambient air which may also be heated. Second inlet(s) 20 and second outlet(s) 22 may be in communication with an ambient environment if ambient air is used as a regenerative fluid(s). Alternatively, second inlet(s) 20 may be connectable to a source of regenerative fluid(s) such as nitrogen, which may be pressurized. Screen 24 may be disposed across second inlet(s) 20 to substantially prevent foreign objects from entering chamber(s) 12 while permitting a flow of regenerative fluid(s) through second inlet(s) 20 and into chamber(s) 12 during the release mode of operation. Screen 26 may be disposed within chamber(s) 12 to substantially retain adsorbent(s) 14 into chamber(s) 12 while still permitting a flow of patient expired gas(es) and/or regenerative fluid(s) through chamber(s) 12.

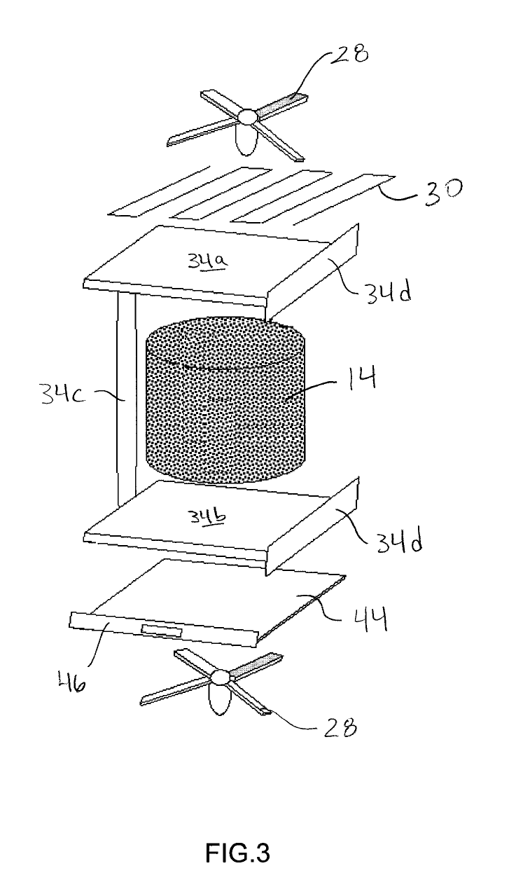

FIG. 2 shows a cross section view of assembly 10 taken along line 2-2 in FIG. 1. FIG. 3 shows an exploded view of some components in assembly 10. Assembly 10 may comprise integral regeneration equipment permitting regeneration of adsorbent(s) 14 on-site and without the need for separate regeneration equipment external to assembly 10. Assembly 10 may be coupled to a source of regeneration fluid(s) such as air and/or nitrogen. The source of regeneration fluid(s) may be pressurized and released through chamber(s) 12 so that a pressure differential between source of regeneration fluid(s) and chamber(s) 12 may serve to induce a flow of regenerative fluid(s) through chamber(s) 12. Alternatively, assembly 10 may comprise one or more integral fluid propellers such as electric fan(s) 28, for example, which may be used to induce a flow of regenerative fluid(s) through chamber(s) 12. Fan(s) 28 may be disposed within or near second inlet(s) 20 and/or second outlet(s) 22. Assembly may also comprise heater(s) 30 configured to introduce heat into chamber(s) 12 during the release mode of operation. Heater(s) 30 may be configured to heat the regenerative fluid(s) before entering or while inside chamber(s) 12. Heater(s) 30 may comprise one or more electric heating elements disposed within or near second inlet(s) 20.

If nitrogen or ambient air is used as a regenerative fluid, it may be desirable that the regenerative fluid be heated to a temperature of around 60° C. to 100° C. for example. A person skilled in the relevant arts will understand that the regeneration time will depend on parameters such as: the type and amount of adsorbent(s); type and amount of the regenerative fluid(s); and the temperature of the regenerative fluid(s) and/or adsorbent(s). Heater(s) 30 and fan(s) 28 may be sized accordingly to supply a desired flow rate of regenerative ambient air at a desired temperature in order to achieve regeneration of adsorbent(s) 14 in a suitable amount of time. For example, the amount of time required for regeneration could be reduced by providing regenerative fluid(s) at a higher flow rate and at a higher temperature as opposed to providing regenerative fluid(s) at a lower flow rate and at a lower temperature.

Assembly 10 may comprise control device(s) for switching from the capture mode of operation to the release mode of operation. Control device(s) may be manually, semi-automatically and/or automatically actuated. For example, assembly 10 may comprise first flow control device(s) 32 for controlling the flow of patient expired gas(es) through chamber(s) 12 and second flow control device(s) 34 for controlling the flow of regenerative fluid(s) through chamber(s) 12.

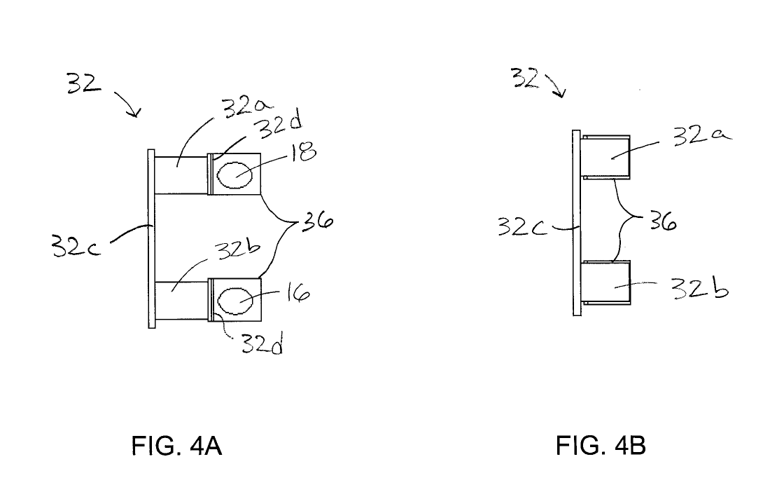

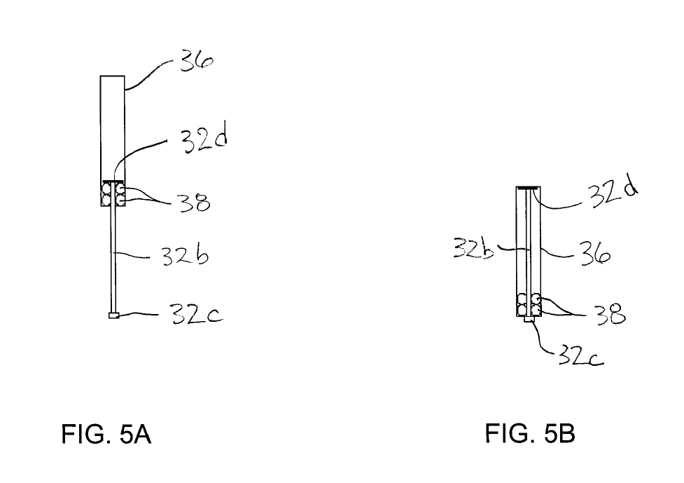

FIGS. 4A and 4B are a cross section of first flow control device(s) 32 taken along line 4-4 in FIG. 1. FIGS. 5A and 5B are a cross section of first flow control device(s) 32 taken along line 5-5 in FIG. 1. First flow control device(s) 32 may comprise movable gates 32a, 32b, common handle 32c connected to both gates 32a, 32b and stop(s) 32d (see FIGS. 4A, 4B, 5A and 5B). Second flow control device(s) 34 may be constructed similarly to first flow control device(s) 32 and may comprise movable gates 34a, 34b, common handle 34c connected to both gates 34a, 34b and stop(s) 34d (see FIGS. 2 and 3).

Gates 32a, 32b of first flow control device(s) 32 may be configured to allow (open position) or at least substantially prevent (closed position) patient expired gas(es) through first inlet(s) 16 and first outlet(s) 18 respectively. Handle 32c may be used as an actuation device to manually move (e.g. slide) both gates 32a and 32b, relative to stationary structure of assembly 10, between the open and closed positions simultaneously in a single action. Gates 32a, 32b may be substantially linearly and/or rotationally movable. For example, gates 32a, 32b may be received and movable within sleeves 36. The movement of one or both gates 32a, 32b may be guided by one or more guide rollers 38 (see FIGS. 5A and 5B). Guide roller(s) 38 may have a cylindrical shape and extend substantially across a width of gates 32a, 32b. One or more guide rollers 38 may be disposed on opposite sides of gates 32a, 32b.

Stop(s) 32d may serve the purpose of defining an open and/or closed position(s) of gates 32a, 32b. For example stop(s) 32d may be disposed on gates 32a, 32b and may interact with stationary structure of assembly 10 such as sleeve(s) 36 in order to prevent further movement of gates 32a, 32b beyond a closed and/or open position(s). Alternatively or in combination, stop(s) 32d may also interact with guide roller(s) 38 to prevent further movement of gates 32a, 32b beyond a closed and/or open position(s). Interaction between stop(s) 32d and wall(s) 36 and/or roller(s) 38 may include physical contact.

Stop(s) 32d may also substantially hinder the inadvertent/accidental opening and closing of gates 32a, 32b. For example, at least a portion of stop(s) 32d, and/or sleeve(s) 36 and/or roller(s) 38 may be constructed from a magnetic material and one of stop(s) 32d, and/or sleeve(s) 36 and/or roller(s) 38 may be magnetized. For example, stop(s) 32d may be magnetized and sleeve(s) 36 and roller(s) 38 may be constructed from magnetic material(s) and thereby be magnetically attracted by magnet(s) incorporated in or part of stop(s) 32d. Accordingly, the interaction between stop(s) 32d and sleeve(s) 36 and/or roller(s) 38 may include a magnetic attraction defining a minimum threshold force to be overcome in order to initiate movement of gates 32a, 32b to/from an open an/or closed position. For example, when gates 32a, 32b are in an open position (see FIGS. 4A and 5A), stop(s) 32d may be in physical contact with or close enough to roller(s) 38 so that a magnetic force must be overcome to initiate movement of gates 32a, 32b towards the closed position. Similarly, when gates 32a, 32b are in a closed position and substantially occluding first inlet(s) 16 and first outlet(s) 18 (see FIGS. 4B and 5B), stop(s) 32d may be in physical contact with or close enough to a wall of sleeve(s) 36 so that a magnetic force must be overcome to initiate movement of gates 32a, 32b towards the open position. In light of the present disclosure, one skilled in the relevant arts will appreciate that other methods and devices could be used instead of or in combination with magnetic stop(s) 32d to substantially hinder inadvertent/accidental opening and closing of gates 32a, 32b.

Further, when gates 32a, 32b are in a closed position and substantially occluding first inlet(s) 16 and first outlet(s) 18 (see FIGS. 4B and 5B), the physical contact and magnetic attraction between stop(s) 32d and wall of sleeve(s) 36 may provide suitable sealing capability (e.g. magnetic seal) to substantially prevent regenerative fluid(s) from entering the breathing circuit via first inlet(s) 16 and first outlet(s) 18. Similarly, when gates 32a, 32b are in an open position (see FIGS. 4A and 5A), the physical contact and magnetic attraction between stop(s) 32d and roller(s) 38 may provide suitable sealing capability (e.g. magnetic seal) to substantially prevent anesthetic gas(es) from leaking into the ambient environment (e.g. operating room) via any clearances that may exist between gates 32a, 32b and sleeve(s) 36 for example. One skilled in the relevant arts will also appreciate that other methods and devices could be used instead of or in combination with magnetic stop(s) 32d to provide suitable sealing capability. For example and alternatively, first inlet(s) 16 and first outlet(s) 18 may be physically disconnected from the breathing circuit and a properly fitted cap (not shown) may be used to occlude each of first inlet(s) 16 and first outlet(s) 18 during regeneration of adsorbent(s) 14.

As shown in FIGS. 2 and 3 and mentioned above, second flow control device(s) 34 may be constructed and operated similarly to first flow control device(s) 32. For example, second flow control device(s) 34 may comprise movable gates 34a, 34b, a common handle 34c connected to both gates 34a, 34b and stop(s) 34d. Gates 34a, 34b may be configured to allow (open position) or at least substantially prevent (closed position) a flow of regenerative fluid(s) through second inlet(s) 20 and second outlet(s) 22 respectively. Handle 34c may be used as an actuating device to manually move (e.g. slide) both gates 34a and 34b, relative to stationary structure of assembly 10, between the open and closed positions simultaneously in a single action. Gates 34a, 34b may be substantially linearly and/or rotationally movable. The movement of one or both gates 34a, 34b may be guided by one or more guide rollers 40 (see FIG. 2). Guide roller(s) 40 may be cylindrical and extend substantially across a width of gates 34a, 34b. One or more guide rollers 40 may be disposed on opposite sides of gates 34a, 34b.

Stop(s) 34d may serve the purpose of defining an open and/or closed position(s) of gates 34a, 34b. For example stop(s) 34d may be disposed on one or both gates 34a, 34b and may interact with stationary structure of assembly 10 such as wall(s) 42 in order to prevent further movement of gates 34a, 34b beyond a closed and/or open position(s). Alternatively or in combination, stop(s) 34d may also interact with guide roller(s) 40 to prevent further movement of gates 34a, 34b beyond a closed and/or open position(s). Interaction between stop(s) 34d and wall(s) 42 and/or roller(s) 40 may include physical contact.

Stop(s) 34d may also substantially hinder the inadvertent/accidental opening and closing of gates 34a, 34b. For example, at least a portion of stop(s) 34d, and/or wall(s) 42 and/or roller(s) 40 may be constructed from a magnetic material and one of stop(s) 34d, and/or wall(s) 42 and/or roller(s) 40 may be magnetized. For example, stop(s) 34d may be magnetized and wall(s) 42 and roller(s) 40 may be constructed from magnetic material and thereby be magnetically attracted by magnet(s) incorporated in or part of stop(s) 34d. Accordingly, the interaction between stop(s) 34d and wall(s) 42 and/or roller(s) 40 may include a magnetic attraction defining a minimum threshold force to be overcome in order to initiate movement of gates 34a, 34b to/from an open an/or closed position. For example, when gates 34a, 34b are in an open position, stop(s) 34d may be in physical contact with or close enough to roller(s) 40 so that a magnetic force must be overcome to initiate movement of gates 34a, 34b towards the closed position. Similarly, when gates 34a, 34b are in a closed position and substantially occluding second inlet(s) 20 and second outlet(s) 22, stop(s) 34d may be in physical contact with or close enough to wall(s) 42 so that a magnetic force must be overcome to initiate movement of gates 34a, 34b towards the open position. Again, in light of the present disclosure, one skilled in the relevant arts will appreciate that other methods and devices could be used instead of or in combination with magnetic stop(s) 34d to substantially hinder inadvertent/accidental opening and closing of gates 34a, 34b.

Assembly 10 may further comprise replaceable filter element(s) 44 (see FIG. 3) which may be configured and positioned within (e.g. integral to) assembly 10 to filter (e.g. trap particulate matter) before regenerative fluid(s) and CO₂ is released via second outlet(s) 22 during the release mode of operation. Filter element(s) 44 may be disposed downstream (during the release mode of operation) from adsorbent(s) 14. Filter element(s) 44 may be inserted (e.g. slid) into and removed from assembly 10 in a linear manner by pushing or pulling handle 46.

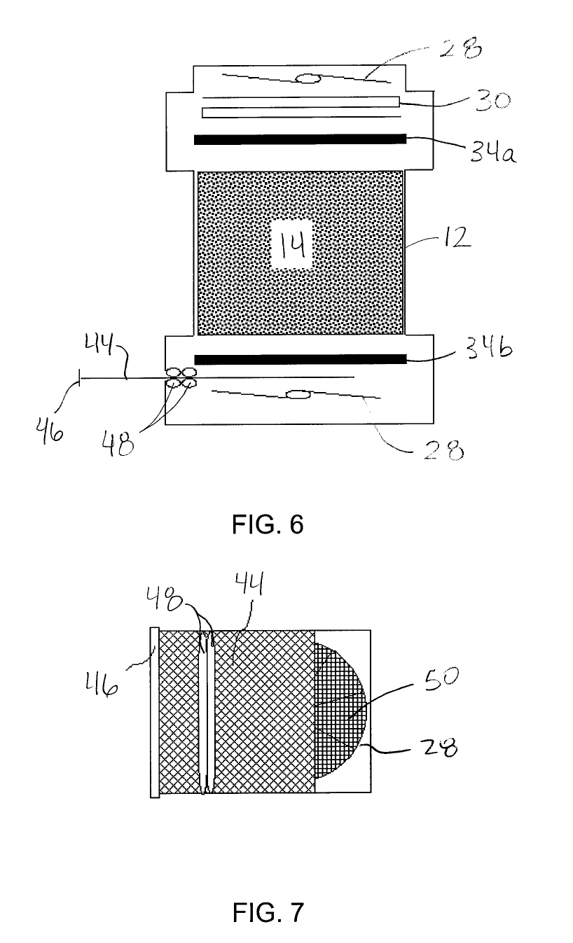

FIG. 6 is a cross-sectional view of the assembly 10 along line 6-6 in FIG. 1 showing filter element(s) 44 partially withdrawn/inserted into assembly 10. The movement of filter element(s) 44 may be guided by guide rollers 48. Alternatively, filter element(s) 44 could be guided by slots (not shown) formed in the housing /structure (e.g. common support structure) of assembly 10. Filter element(s) 44 may be of a type selected based on the type of adsorbent(s) 14. For example, filter element(s) 44 may be a High-Efficiency Particulate Air (HEPA) filter element configured to capture approximately 99% of all particles greater than 0.3 micrometers.

FIG. 7 is top view of filter element(s) 44 partially withdrawn/inserted into assembly 10, hence partially extending across (e.g. partially occluding) passage 50 of second outlet(s) 22 in which fan(s) 28 may be disposed.

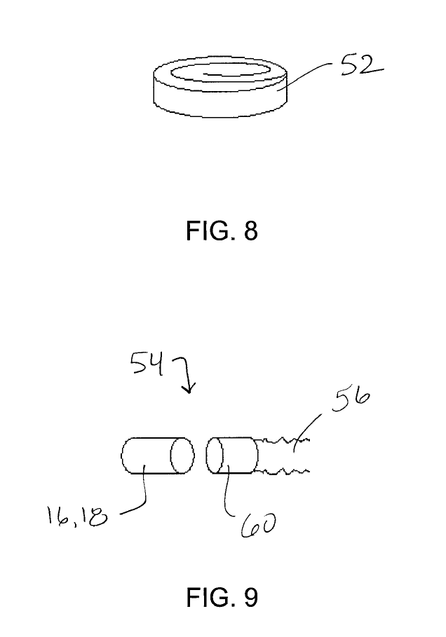

FIG. 8 shows fluid distributor(s) 52 which may be included in assembly 10 and cause the flow of patient expired gas(es) entering chamber(s) 12 to be substantially distributed across adsorbent(s) 14. For example, distributor(s) 52 may be configured to induce a substantially helical movement in the flow of patient expired gas(es) so that the flow of patient expired gas(es) entering chamber(s) 12 may be substantially evenly distributed across adsorbent(s) 14.

FIG. 9 shows arrangement 54 for coupling first inlet(s) 16 and/or first outlet(s) 18 in-line with expiratory limb 56, part of a breathing circuit of anesthesia machine 58 (see FIG. 10). Arrangement 54 may comprise fitting(s) 60 adapted to couple directly to first inlet(s) 16 and/or first outlet(s) 18. Fitting(s) 60 may be configured to be easily inserted and removed from first inlet(s) 16 and/or first outlet(s) 18 by sliding (e.g. inserting) and retaining fitting(s) 60 into first inlet(s) 16 and/or first outlet(s) 18. Fitting(s) 60 and corresponding first inlet(s) 16 and/or first outlet(s) 18 may have cooperating circular cross-sections and may be configured and dimensioned so that a pressure fit, producing a retaining force, is established when fitting(s) 60 is(are) inserted into first inlet(s) 16 and/or first outlet(s) 18. Accordingly, fitting(s) 60 may have an axially tapered outer surface and/or first inlet(s) 16 and/or first outlet(s) 18 may have an axially tapered inner surface configured to establish the pressure fit between the mating parts. Alternatively, arrangement 54 may be configured such that first inlet(s) 16 and/or first outlet(s) 18 may be inserted and retained into fitting(s) 60 in a similar fashion. Additional means for providing a positive engagement (not shown) between fitting(s) 60 and first inlet(s) 16 and/or first outlet(s) 18 may also be provided.

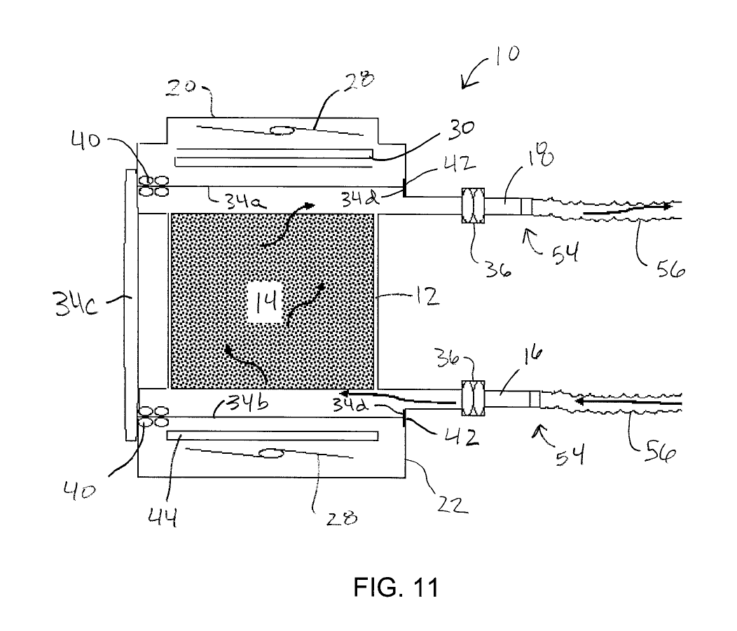

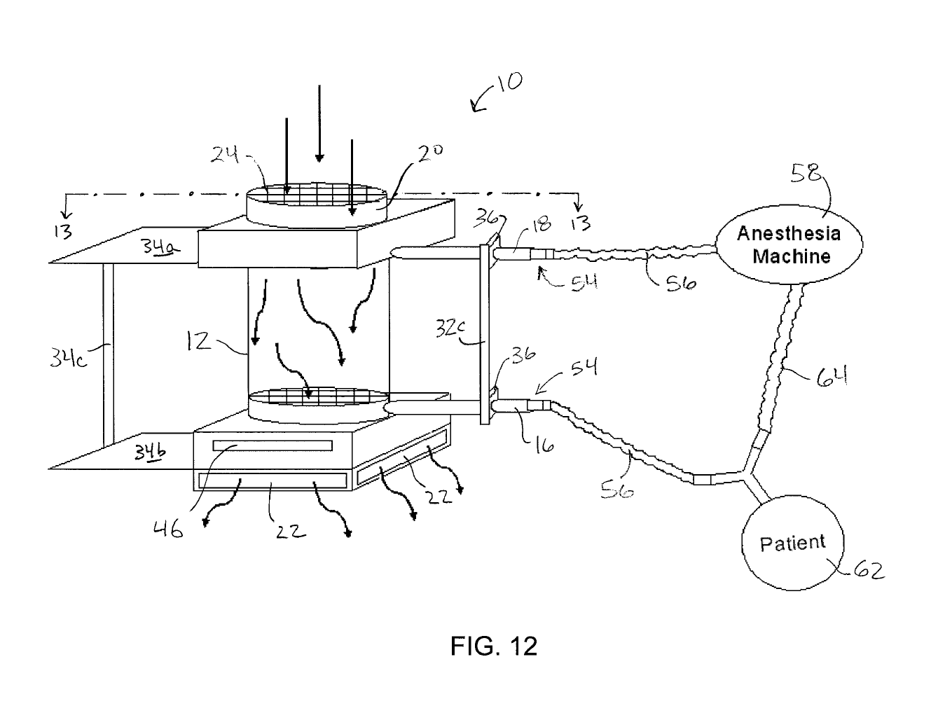

FIG. 10 shows assembly 10 connected with anesthesia machine 58 and configured to operate in a capture mode of operation. FIG. 11 shows a cross-sectional view of assembly 10 taken along line 11-11 in FIG. 10. Arrows in FIGS. 10 and 11 illustrate a flow of gas(es) through an anesthetic breathing circuit. Assembly 10 may be external to anesthesia machine 28 or may be integrated to anesthesia machine 28. For example, an existing canister (not shown) of a conventional or other type of anesthesia machine 28 may be substituted by assembly 10. Alternatively, assembly 10 may be integrated into expiratory limb 56 of the breathing circuit which may further include an existing canister (not shown) of a conventional or other type of anesthesia machine 28 where the existing canister may be empty and thereby allow a continuous flow of gas(es) without the need for modification of current anesthesia breathing machines/systems. In any event, assembly 10 may be standalone or self-contained so that, for example, no auxiliary regenerative equipment may be required and hence regeneration of adsorbent(s) 14 may be conducted on-site. If ambient air is used as a regenerative fluid, no separate source of regenerative fluid(s) (e.g. pressurized air or nitrogen) may be required.

During a capture mode of operation of assembly 10, patient(s) 62 may receive oxygen (O₂) and anesthetic gas(es) from anesthesia machine(s) 58 via inspiratory limb(s) 64 of the breathing circuit and may expire gas(es) through expiratory limb(s) 56. For example, the anesthetic gas(es) may comprise one or more of desflurane, enflurane, halothane, isoflurane and sevoflurane. The expired gas(es) from patient(s) 62 may contain CO₂ and anesthetic gas(es). Assembly(ies) 10 may be connected in-line with expiratory limb(s) 56. Assembly(ies) 10 may receive expired gas(es) from patient(s) 62, treat the patient expired gas(es) and return the treated patient expired gas(es) to anesthesia machine(s) 58 for further treatment as necessary and recycling (e.g. re-breathing). The treating of patient expired gas(es) by assembly(ies) 10 may comprise the removal of CO₂ from the patient expired gas(es).

During the capture mode of operation, assembly(ies) 10 may be configured to received patient expired gas(es) into chamber(s) 12, remove CO₂ from the patient expired gas(es) by adsorption using adsorbent(s) 14 and return treated patient expired gas(es) to anesthesia machine(s) 58. During this mode of operation, first flow control device(s) 32 may be configured to permit flow of patient expired gas(es) through first inlet(s) 16, into chamber(s) 12 and out of first outlet(s) 18. Specifically, gates 32a and 32b may be in the open position as shown in FIGS. 4A and 5A. Also, second flow control device(s) 34 may be in a closed position such that gates 34a and 34b are positioned to substantially prevent fluid flow through second inlet(s) 20 and second outlet(s) 22 by occluding second inlet(s) 20 and second outlet(s) 22 respectively as shown in FIG. 11. Stop(s) 34d may be in contact and/or magnetic interaction with wall(s) 42 and provide a retention force holding gates 34a, 34b in the closed position. Regeneration equipment such as fan(s) 28 and heater(s) 30 may be deactivated since they may not be required during a capture mode of operation.

Following a certain period of operation in the capture mode, adsorbent(s) 14 may become loaded with captured CO₂ and require regeneration before continued and/or repeated use(s). Accordingly, assembly(ies) 10 may be switched from the capture mode of operation to a release mode of operation. If the need for regeneration of adsorbent(s) 14 occurs during a procedure where anesthesia is still required, assembly(ies) 10 requiring regeneration may be taken offline and replaced by one or more additional assemblies 10 added to the breathing circuit so as to avoid interruption of the procedure and/or anesthesia. Since the regeneration equipment required for regeneration of adsorbent(s) 14 may be integral to assembly(ies) 10, the release operation may be conducted on-site and assembly(ies) 10 may not need to be sent to another facility for regeneration. The release operation may also be conducted while assembly(ies) 10 remains physically connected to anesthesia machine(s) 58 via expiratory limb(s) 56 but not in communication with expiratory limb(s) 56. It is, of course, understood that regeneration of adsorbent 14 may be effected while the machine 58 is not being used to treat a patient 62.

While it is intended that regeneration of adsorbent(s) 14 be conducted on-site, it may be necessary or desirable to send assembly 10 to another facility to have adsorbent(s) 14 replaced after a number of capture/regeneration cycles. For example, depending on the type of adsorbent(s) 14 used and the operating conditions, adsorbent(s) 14 may eventually become exhausted such that the performance of adsorbent(s) 14 may become reduced after a number (e.g. 100 or more) capture/regeneration cycles. Accordingly, after a certain period of operation, it may be desirable to replace adsorbent(s) 14 with newer and/or more efficient material(s), which may be of the same or different type(s).

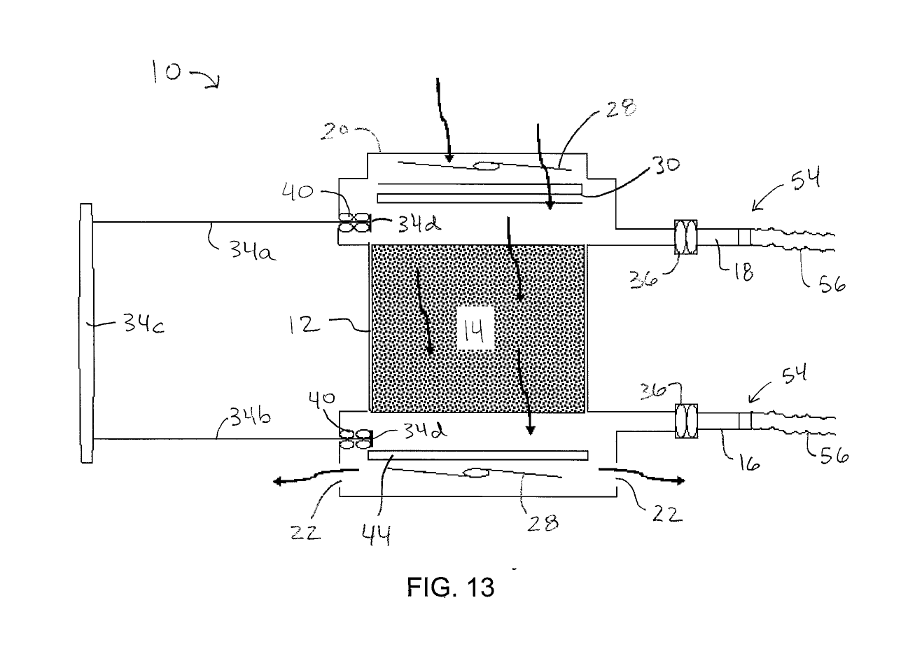

FIG. 12 shows assembly 10 configured to operate in the release mode of operation while still being connected to the breathing circuit (i.e 64, 62, 56) of the anesthesia machine 58. FIG. 13 shows a cross-sectional view of assembly 12 taken along line 13-13 in FIG. 12. Arrows in FIGS. 12 and 13 illustrate a flow of regenerative fluid(s) through assembly 10.

During the release mode of operation, assembly(ies) 10 may be taken offline to no longer receive patient expired gas(es) and regenerative fluid(s) may be introduced into chamber(s) 12 to come into contact with adsorbent(s) 14 to cause the adsorbed CO₂ to be released from adsorbent(s) 14 and thereby regenerate adsorbent(s) 14. Depending on the type of adsorbent(s) 14 used, regenerative fluid(s) may include a fluid comprising of pure nitrogen (99.99% or higher) and/or air such as ambient air for example. During this mode of operation, first flow control device(s) 32 may be configured to substantially prevent fluid flow through first inlet(s) 16 and first outlet(s) 18 by occluding first inlet(s) 16 and first outlet(s) 18 respectively. Specifically, gates 32a and 32b may be in the closed position as shown in FIGS. 4B and 5B. Also, second flow control device(s) 34 may be in the open position such that gates 34a and 34b may permit fluid flow through second inlet(s) 20 and second outlet(s) 22 respectively as shown in FIGS. 4A and 5A. In the open position, stop(s) 34d may be in contact and/or magnetic interaction with guide roller(s) 40 and provide a retention force holding gates 34a, 34b in the open position.

Regeneration equipment integral to assembly 10 may be used to regenerate adsorbent(s) 14. For example, when ambient air is used as a regenerative fluid, fan(s) 28 may be used to induce a flow of ambient air into second inlet(s) 20, through chamber(s) 12 and out of second outlet(s) 22. Heater(s) 30 may also be used to introduce heat into chamber(s) 12 to promote regeneration of adsorbent(s) 14. Heater(s) 30 may be disposed near second inlet(s) 20 to add heat to the regenerative fluid(s) (e.g. air) before it enters chamber(s) 12 if required. If the regenerative fluid(s) is(are) provided at a suitable temperature, the use of heater(s) 30 may not be required. As the regenerative fluid(s) contacts adsorbent(s) 14, it may cause the loaded adsorbent(s) 14 to release the previously captured CO₂ and caused the CO₂ to be released together with the regenerative fluid(s) through second outlet(s) 22. When ambient air is used as a regenerative fluid, CO₂ may be released into the ambient environment. Filter(s) 44 may be used to substantially remove particulate matter that may have otherwise been carried out by the regenerative fluid exiting assembly 10 via second outlet(s) 22. Filter(s) 44 may be replaceable, recyclable/washable and/or disposable and may be replaced as required.

Assembly 10 may comprise one or more fan(s) 28 disposed near second inlet(s) 20 (e.g. upstream from adsorbent(s) 14) for pushing regenerative fluid(s) such as ambient air into chamber(s) 12 and through the bed of adsorbent(s) 14. Assembly 10 may also comprise one or more fan(s) 28 disposed near second outlet(s) 22 (e.g. downstream from adsorbent 14) for pulling regenerative fluid out of the bed of adsorbent(s) 14 (e.g. create a vacuum relative to the pressure in chamber(s) 12).

Control device(s) such as, for example, flow control device(s) 32, 34 of assembly 10 for switching from the capture mode of operation to the release mode of operation or vice-versa may be manually, semi-automatically or automatically actuated. For example, in switching from the capture mode of operation to the release mode of operation, an operator may manually: (1) push on handle 32c and in a single action cause first flow control device(s) 32 to move from an open position to a closed position; (2) pull on handle 34c and in a single action cause second flow control device(s) 34 to move from a closed position to an open position; and (3) activate at least some of the regeneration equipment that may be integral to assembly 10. In switching from the release mode of operation to the capture mode of operation, an operator may, for example, manually: (1) de-activate at least some of the regeneration equipment; (2) push on handle 34c and in a single action cause second flow control device(s) 34 to move from an open position to a closed position; and (3) pull on handle 32c and in a single action cause first flow control device(s) 32 to move from a closed position to an open position.

Alternatively, control device(s) of assembly 10 may be configured for a more automated switching between the capture mode of operation and the release mode of operation.

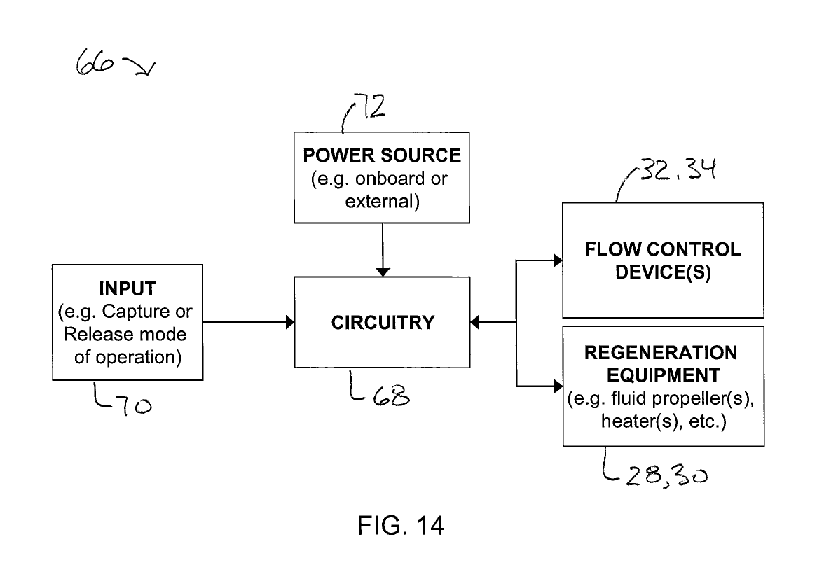

FIG. 14 shows an example of an arrangement, generally shown at 66, for semi-automatically and/or automatically switching assembly 10 between the capture mode of operation and the release mode of operation. Arrangement 66 may comprise circuitry 68 configured to receive input(s) 70 and appropriately configure flow control device(s) 32, 34 and regeneration equipment 28, 30 according to the input(s) 70 received. Circuitry 68 may be integral to assembly 10 and may be coupled to power source(s) 72 which may also be integral to assembly 10 (e.g. onboard battery) or external to assembly 10. Input(s) 70 may be an electric or electronic signal representative of the desired mode of operation. For example, input(s) 70 may originate from a user selecting a mode of operation (e.g. capture or release) by actuating a switch, button or other types of hardware and/or software selection means. Alternatively, in the event where assembly 10 is integrated within anesthesia machine(s) 58, input(s) 70 may originate from control circuitry of anesthesia machine(s) 58 which may be configured to detect/determine when a change in operating mode is required. Flow control device(s) 32, 34 may be electrically operated and powered by power source(s) 72 via circuitry 68. For example, assembly 10 may comprise one or more solenoids or other types of actuators such as electrically powered actuators that may be used to move gates 32a, 32b, 34a, 34b between the open and closed positions. Similarly, regeneration equipment 28, 30 may also be powered by power source(s) 72 via circuitry 68. Circuitry 68 may also be configured to receive feedback representative of the state of flow control device(s) 32, 34 and/or regeneration equipment 28, 30 via suitable sensor(s).

In light of a review of this disclosure, it will be apparent to those skilled in the art that assembly 10 may be constructed using materials and methods used in the construction of conventional or other types of devices used in similar processes. For ease of integration into existing or new anesthesia machine(s) 58 or systems, assembly(ies) 10 may be constructed using materials compatible with and typically used with such processes and substances. Assembly(ies) 10 may also have an overall envelope and number/types of inputs and outputs such that assembly(ies) 10 may be compatible with existing anesthesia machines and readily replace existing canisters in such anesthesia machines. Chamber(s) 12, fluid passages and coupling arrangements 54 within assembly 10 may be substantially sealed to prevent any harmful dust(s) or chemical(s), such as dust caused by filling and emptying chamber(s) 12 or byproducts produced through interactions with the anesthetic and adsorbent(s) 14, from coming in contact with patients and/or healthcare workers.

The above description is meant to be exemplary only, and one skilled in the art will recognize that changes may be made to the embodiments described without departing from the scope of the invention disclosed. Still other modifications which fall within the scope of the present invention will be apparent to those skilled in the art, in light of a review of this disclosure, and such modifications are intended to fall within the appended claims.

1. A standalone assembly for removing carbon dioxide from patient expired gas during anesthesia, the assembly comprising:
a chamber configured to contain an adsorbent for treating the patient expired gas, the chamber including a first inlet for receiving the patient expired gas for treatment by the adsorbent and a first outlet for returning treated patient expired gas to an anesthesia machine during a capture mode of operation;
regeneration equipment configured to regenerate the adsorbent during a release mode of operation; and
a control device for switching from the capture mode of operation to the release mode of operation.

2. The standalone assembly as defined in claim 1, wherein the integral regeneration equipment is configured to induce a flow of regenerative fluid for regenerating the adsorbent.

3. The standalone assembly as defined in claim 2, wherein the integral regeneration equipment is configured to introduce heat into the chamber.

4. The standalone assembly as defined in claim 1, wherein the integral regeneration equipment comprises a fan to induce a flow of ambient air into the chamber.

5. The standalone assembly as defined in claim 4, wherein the integral regeneration equipment comprises a heater to heat the flow of ambient air.

6. The standalone assembly as defined in claim 1, wherein the chamber comprises a second inlet for receiving a regenerative fluid for regenerating the adsorbent and a second outlet for releasing the regenerative fluid, after the regenerative fluid has at least partially regenerated the adsorbent, during the release mode of operation.

7. The standalone assembly as defined in claim 6, wherein the second inlet is configured to be in communication with a source of regenerative fluid.

8. The standalone assembly as defined in claim 6, wherein the second inlet and the second outlet are in communication with an ambient environment during the release mode of operation.

9. The standalone assembly as defined in claim 6, wherein the integral regeneration equipment is configured to: induce a flow of regenerative fluid through the chamber via the second inlet and second outlet; and heat the flow of regenerative fluid.

10. (canceled)

11. The standalone assembly as defined in claim 9, wherein the control device comprises a single-action flow control device to simultaneously occlude the first inlet and the first outlet.

12. The standalone assembly as defined in claim 9, wherein the control device comprises a single-action flow control device to simultaneously open the second inlet and the second outlet.

13. The standalone assembly as defined in claim 9, wherein the integral regeneration equipment comprises a first fluid propeller disposed upstream of the adsorbent and a second fluid propeller disposed downstream of the adsorbent.

14. The standalone assembly as defined in claim 2, comprising a filter for filtering the regenerative fluid following contact of the regenerative fluid with the adsorbent.

15. A method for removing carbon dioxide from patient expired gas during anesthesia using a standalone assembly, the assembly comprising a chamber containing an adsorbent and regeneration equipment for regenerating the adsorbent, the method comprising:
during a capture mode of operation of the assembly: receiving the patient expired gas into the chamber; removing carbon dioxide from the patient expired gas using the adsorbent; and returning treated patient expired gas to an anesthesia machine;
switching from a capture mode of operation to a release mode of operation; and
activating the regeneration equipment integral to the assembly to regenerate the adsorbent.

16. The method as defined in claim 15, wherein the activation of the regeneration equipment includes activating a fluid propeller integral to the assembly to induce a flow of regenerative fluid in the chamber.

17. The method as defined in claim 15, wherein the activation of the regeneration equipment includes activating a heater integral to the assembly to introduce heat into the chamber.

18. The method as defined in claim 15, comprising directing a flow of nitrogen through the chamber to regenerate the adsorbent.

19-39. (canceled)

40. A system for removing carbon dioxide from patient expired gas during anesthesia, the assembly comprising:
a chamber configured to contain an adsorbent for treating the patient expired gas, the chamber including a first inlet for receiving the patient expired gas for treatment by the adsorbent and a first outlet for returning treated patient expired gas to an anesthesia machine during a capture mode of operation;
regeneration equipment configured to regenerate the adsorbent during a release mode of operation, the regeneration device and the chamber being integrated in a common support structure; and
a control device for switching from the capture mode of operation to the release mode of operation.

41. The system as defined in claim 40, wherein the regeneration equipment comprises a heater for introducing heat into the chamber.

42. The system as defined in claim 41, wherein the regeneration equipment comprises a fluid propeller configured to induce a regenerative fluid through the chamber during a release mode of operation.

43-48. (canceled)
